(19) United States
(12) Patent Application Publication
(10) Pub. No.: US 2019/0274552 A1
(43) Pub. Date: Sep. 12, 2019

(54) APPARATUS AND METHOD FOR MEASURING BLOOD PRESSURE

(71) Applicants: SAMSUNG ELECTRONICS CO., LTD., Suwon-si (KR); UNIVERSITY OF MARYLAND, COLLEGE PARK, College Park, MD (US)

(72) Inventors: Dae Geun Jang, Yongin-si (KR); Youn Ho Kim, Hwaseong-si (KR); Peyman Yousefian, Jersey City, NJ (US); Jin-Oh Hann, Potomac, MD (US); Sungtae Shin, Greenbelt, MD (US); Azin Sadat Mousavi, College Park, MD (US)

(73) Assignees: SAMSUNG ELECTRONICS CO., LTD., Suwon-si (KR); UNIVERSITY OF MARYLAND, COLLEGE PARK, College Park, MD (US)

(21) Appl. No.: 16/298,527

(22) Filed: Mar. 11, 2019

(30) Foreign Application Priority Data
Mar. 12, 2018 (KR) 10-2018-0028683

(51) Int. Cl. A61B 5/021; A61B 5/11; A61B 5/00

(52) U.S. Cl. CPC A61B 5/021; A61B 5/1102; A61B 5/7278; A61B 2562/0252; A61B 5/721; A61B 5/681; A61B 2562/0219; A61B 5/7253

(57) ABSTRACT

An apparatus for measuring blood pressure according to one aspect may include a limb ballistocardiogram (BCG) sensor configured to attach to a limb of a user and measure a limb BCG signal of the user, and a processor configured to extract blood pressure-related features from the measured limb BCG signal and estimate blood pressure of the user based on at least part of the extracted blood pressure-related features.

### CROSS-REFERENCE TO RELATED APPLICATION(S)

This application claims priority from Korean Patent Application No. 10-2018-0028683, filed on Mar. 12, 2018, in the Korean Intellectual Property Office, the entire disclosure of which is incorporated herein by reference in its entirety.

### BACKGROUND

### 1. Field

Apparatuses and methods consistent with exemplary embodiments relate to a cuffless blood pressure measurement technology.

### 2. Description of Related Art

Healthcare technology has attracted much attention due to the society's increasingly aging population and relevant social problems that accompany such societal changes, such as increase in medical expenses. Accordingly, medical devices that can be utilized by hospitals and inspection agencies as well as small-sized medical devices that can be carried by individuals such as wearable devices have been developed. In addition, a small-sized medical device may be worn by a user in the form of a wearable device capable of directly measuring cardiovascular health indicators such as a blood pressure or the like, so that the user can measure and manage her cardiovascular health status.

Therefore, recently, studies have been actively conducted on methods of estimating blood pressure by analyzing a bio-signal for the purpose of miniaturizing the size of a device and improving the accuracy of blood pressure estimation.

### SUMMARY

One or more exemplary embodiments provide an apparatus and method for measuring blood pressure.

According to an aspect of an exemplary embodiment, there is provided an apparatus for measuring blood pressure, including: a limb ballistocardiogram (BCG) sensor configured to attach to a limb of a user and measure a limb BCG signal of the user; and a processor configured to extract blood pressure-related features from the measured limb BCG signal and estimate blood pressure of the user based on at least part of the extracted blood pressure-related features.

The limb BCG sensor may include at least one of an acceleration sensor, a load cell sensor, a polyvinylidene fluoride (PVDF) film sensor, and an electro mechanical film (EMFi) sensor.

The processor may include: a signal transformer configured to transform the measured limb BCG signal into a form of a whole-body BCG signal; a signal segmenter configured to segment the transformed limb BCG signal by each period to create a limb BCG signal segment; a feature extractor configured to extract at least one of the blood pressure-related features from the limb BCG signal segment; and a blood pressure estimator configured to estimate the blood pressure of the user based on the extracted at least one of the blood pressure-related features.

The signal transformer may be further configured to transform the measured limb BCG signal into the form of the whole-body BCG signal using at least one of an integrator and a personalized model that defines a relationship between the limb BCG signal and the whole-body BCG signal.

The feature extractor may be configured to extract characteristic points from the limb BCG signal segment and extract the at least one of the blood pressure-related features based on at least one of time intervals between the extracted characteristic points and amplitudes of the extracted characteristic points.

The feature extractor may be further configured to extract a maximum point and a minimum point of the limb BCG signal segment as the characteristic points.

The feature extractor may be further configured to determine a representative signal that represents the transformed limb BCG signal using the limb BCG signal segment and extract the at least one of the blood pressure-related features from the determined representative signal.

The processor may further include a preprocessor configured to remove noise from the measured limb BCG signal.

The processor may include: a signal segmenter configured to segment the measured limb BCG signal by each period to create a limb BCG signal segment; a feature extractor configured to extract at least one of the blood pressure-related features from the limb BCG signal segment; an independent feature extractor configured to extract at least one independent blood pressure-related feature from the extracted at least one of the blood pressure-related features; and a blood pressure estimator configured to estimate blood pressure of the user based on the extracted at least one independent blood pressure-related feature.

The independent feature extractor may be further configured to extract the at least one independent blood pressure-related feature from the extracted at least one of the blood pressure-related features using a dimensionality reduction method.

The processor may include: a signal transformer configured to transform the measured limb BCG signal into a form of a whole-body BCG signal; a signal segmenter configured to segment the transformed limb BCG signal by each period to create a limb BCG signal segment; a feature extractor configured to extract at least one of the blood pressure-related features from the limb BCG signal segment; an independent feature extractor configured to extract at least one independent blood pressure-related feature from the extracted at least one of the blood pressure-related features; and a blood pressure estimator configured to estimate blood pressure of the user based on the extracted at least one independent blood pressure-related feature.

According to an aspect of an exemplary embodiment, there is provided a method of measuring blood pressure, including: measuring a limb BCG signal of a user; extracting blood pressure-related features from the measured limb BCG signal; and estimating blood pressure of the user based on at least part of the extracted blood pressure-related features.

The extracting the blood pressure-related features may include: transforming the measured limb BCG signal into a form of a whole-body BCG signal; segmenting the transformed limb BCG signal by each period to create a limb BCG signal segment; extracting at least one of the blood pressure-related features from the limb BCG signal segment; and estimating blood pressure of the user based on the extracted at least one of the blood pressure-related features.

The transforming the measured limb BCG signal may include transforming the measured limb BCG signal into the form of the whole-body BCG signal using at least one of an integrator and a personalized model that defines a relationship between the limb BCG signal and the whole-body BCG signal.

The extracting the at least one of the blood pressure-related features may include extracting characteristic points from the limb BCG signal segment and extracting the at least one blood pressure-related features based on at least one of time intervals between the extracted characteristic points and amplitudes of the extracted characteristic points.

The extracting the characteristic points may include extracting a maximum point and a minimum point of the limb BCG signal segment as the characteristic points.

The extracting the at least one of the blood pressure-related features may include determining a representative signal that represents the transformed limb BCG signal using the limb BCG signal segment and extracting the at least one of the blood pressure-related features from the determined representative signal.

The extracting the blood pressure-related features may include segmenting the measured limb BCG signal by each period to generate a limb BCG signal segment; extracting at least one of the blood pressure-related features from the limb BCG signal segment; and extracting at least one independent blood pressure-related feature from the extracted at least one of the blood pressure-related features; and estimating blood pressure of the user based on the extracted at least one independent blood pressure-related feature.

The at least one independent blood pressure-related feature may be extracted using a dimensionality reduction method.

The extracting the blood pressure-related features may include: transforming the measured limb BCG signal into a form of a whole-body BCG signal; segmenting the transformed limb BCG signal by each period to create a limb BCG signal segment; extracting at least one of the blood pressure-related features from the limb BCG signal segment; and extracting at least one independent blood pressure-related feature from the extracted at least one of the blood pressure-related features.

Other features and aspects will be apparent from the following detailed description, the drawings, and the claims.

### BRIEF DESCRIPTION OF THE DRAWINGS

The above and/or other aspects will be more apparent by describing certain exemplary embodiments, with reference to the accompanying drawings, in which:

FIG. 1 is a graph showing examples of a whole-body ballistocardiogram (BCG) signal and a limb BCG signal;

FIG. 2 is a block diagram illustrating an apparatus for measuring blood pressure according to an exemplary embodiment;

FIG. 3 is a block diagram illustrating a processor according to an exemplary embodiment;

FIG. 4 is a graph for describing characteristic points;

FIG. 5 is a block diagram illustrating a processor according to another exemplary embodiment;

FIG. 6 is a block diagram illustrating a processor according to still another exemplary embodiment;

FIG. 7 is a flowchart illustrating a method of measuring blood pressure according to an exemplary embodiment;

FIG. 8 is a flowchart illustrating a process of estimating blood pressure according to an exemplary embodiment;

FIG. 9 is a flowchart illustrating a process of estimating blood pressure according to another exemplary embodiment;

FIG. 10 is a flowchart illustrating a process of estimating blood pressure according to still another exemplary embodiment;

FIG. 11 is a block diagram illustrating an apparatus for measuring blood pressure according to another exemplary embodiment; and

FIG. 12 is a diagram illustrating a wrist-wearable device.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they might obscure the description with unnecessary detail.

It should be noted that in some alternative implementations, the functions, steps, actions noted in the blocks may occur out of the order noted in the flowcharts. For example, two blocks shown in succession may in fact be executed substantially concurrently or the blocks may sometimes be executed in the reverse order, depending upon the functionality or actions involved.

Terms described in below are selected by considering functions in the embodiment and meanings may vary depending on, for example, a user or operator's intentions or customs. Therefore, in the following embodiments, when terms are specifically defined, the meanings of terms should be interpreted based on definitions, and otherwise, should be interpreted based on general meanings recognized by those skilled in the art.

As used herein, the singular forms are intended to include the plural forms as well, unless the context clearly indicates otherwise. It will be further understood that the terms “comprises” and/or “comprising,” or “includes” and/or “including” when used in this description, specify the presence of stated features, numbers, steps, operations, elements, components or combinations thereof, but do not preclude the presence or addition of one or more other features, numbers, steps, operations, elements, components or combinations thereof.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

It will also be understood that the elements or components in the following description are discriminated in accordance with their respective main functions. In other words, two or more elements may be made into one element or one element may be divided into two or more elements in accordance with a subdivided function. Additionally, each of the elements in the following description may perform a part or whole of the function of another element as well as its main function, and some of the main functions of each of the elements may be performed exclusively by other elements. Each element may be realized in the form of a hardware component (e.g., circuits, microchips, processors, etc.), a software component (e.g., instructions, programs, applications, firmware, etc.), and/or a combination thereof.

Throughout the drawings and the detailed description, unless otherwise described, the same drawing reference numerals will be understood to refer to the same elements, features, and structures. The relative size and depiction of these elements may be exaggerated for clarity, illustration, and convenience.

The word “exemplary” is used herein to mean “serving as an example or illustration.” Any aspect or design described herein as “exemplary” is not to be construed as preferred or advantageous over other aspects or designs.

A whole-body ballistocardiogram (BCG) signal described in the present description refers to a vibration signal of the body which is caused by the heart rate, and a limb BCG signal may represent a skin vibration signal of the limbs or other body parts (e.g., wrists, ankles, a neck, forearms, etc.).

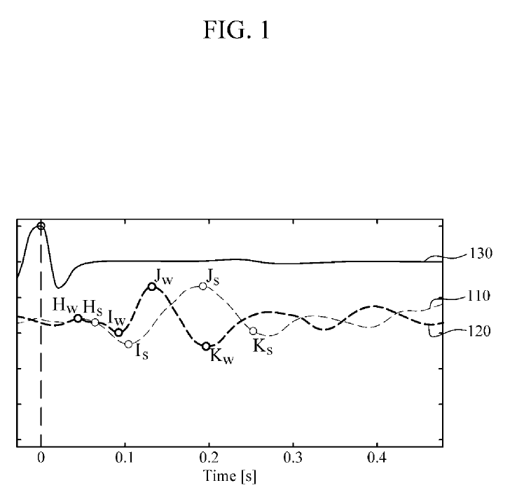

FIG. 1 is a graph showing examples of a whole-body BCG signal and a limb BCG signal. In FIG. 1, the limb BCG signal 120 may be a wrist skin vibration signal measured at a wrist.

Referring to FIG. 1, it can be seen that the whole-body BCG signal 110 and the limb BCG signal 120 have similar characteristic points (e.g., H, I, J, K, and the like), but exhibit different characteristics due to channel characteristics (e.g., compliant human body and the like). For example, as shown in FIG. 1, it can be seen that, when the whole-body BCG signal 110 and the limb BCG signal 120 are beat-gated by an R-wave of an electrocardiogram (ECG) signal, characteristic points of the limb BCG signal 120 appear to be trailed by the whole-body BCG signal 110 and the time difference in which mutually corresponding characteristic points appear increases as the time elapses.

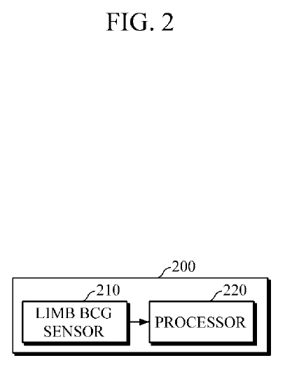

FIG. 2 is a block diagram illustrating an apparatus for measuring blood pressure according to an exemplary embodiment.

The apparatus 200 of FIG. 2 for measuring blood pressure may be implemented by a software module or manufactured in the form of a hardware chip and may be mounted in an electronic device. The electronic device may be a mobile phone, a smartphone, a tablet computer, a notebook computer, a personal digital assistant (PDA), a portable multimedia player (PMP), a navigation system, an MP3 player, a digital camera, a wearable device, and the like. The wearable device may be of a wristwatch type, a wrist band type, a belt type, a necklace type, an ankle band type, a thigh band type, a forearm band type, and the like. However, the electronic device and the wearable device are not limited to the above examples.

Referring to FIG. 2, the apparatus 200 may include a limb BCG sensor 210 and a processor 220.

The limb BCG sensor 210 may be attached to a limb or other body part of a user and may measure a limb BCG signal of the user. To this end, the limb BCG sensor 210 may include various types of sensors, such as an acceleration sensor, a load cell sensor, a polyvinylidene fluoride (PVDF) film sensor, and an electro mechanical film (EMFi) sensor, and the like. The limb or other body parts may include a wrist, an ankle, a neck, a forearm, and the like.

The processor 220 may control an overall operation of the apparatus 200.

The processor 220 may periodically, or when a specific event such as a user command occurs, measure a limb BCG signal of the user by driving the limb BCG sensor 210.

The processor 220 may extract features related to blood pressure by analyzing the limb BCG signal measured by the limb BCG sensor 210, and estimate the user's blood pressure based on all or part of the extracted blood pressure-related features.

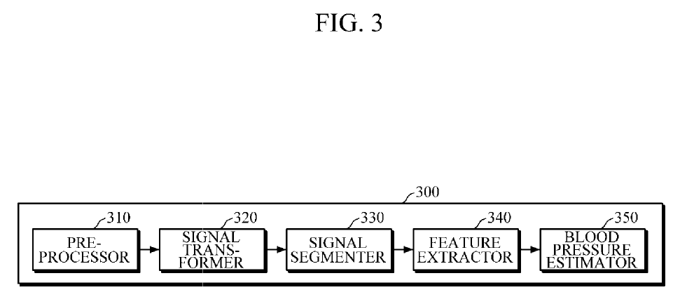

Hereinafter, the processor 220 of the present disclosure will be described in detail with reference to FIGS. 3 to 6.

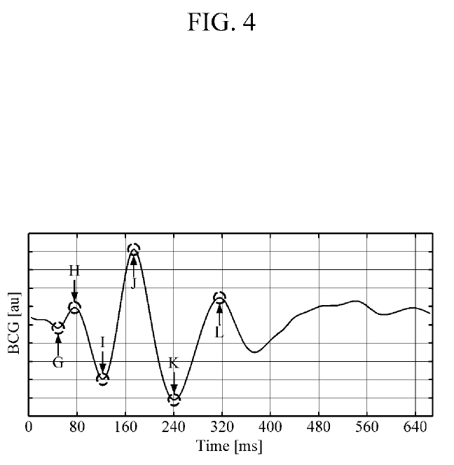

FIG. 3 is a block diagram illustrating a processor according to an exemplary embodiment, and FIG. 4 is a graph for describing characteristic points. The processor 300 of FIG. 3 may be an exemplary embodiment of the processor 220 of FIG. 2.

Referring to FIG. 3, the processor 300 may include a preprocessor 310, a signal transformer 320, a signal segmenter 330, a feature extractor 340, and a blood pressure estimator 350. The various components and elements shown in FIG. 3 and other figures may be implemented with hardware, software, or a combination of both.

The preprocessor 310 may remove noise from a limb BCG signal. In this case, the preprocessor 310 may remove noise from the limb BCG signal using various noise removal techniques, such as filtering, smoothing, and the like.

The signal transformer 320 may transform the limb BCG signal into the form of a whole-body BCG signal.

According to an exemplary embodiment, the signal transformer 320 may transform the limb BCG signal into the form of whole-body BCG signal using a transfer function, such as an integrator or a differentiator. In this case, the type of transfer function may be determined according to the type of a sensor that measures the limb BCG signal (or a form (e.g., displacement, velocity, or acceleration) of the limb BCG signal. For example, when the limb BCG signal is measured by an acceleration sensor, the limb BCG signal may be transformed into the form of a whole-body BCG signal by integrating the limb BCG signal twice using an integrator.

According to another exemplary embodiment, the signal transformer 320 may transform the limb BCG signal into the form of a whole-body BCG signal using a personalized transfer function. In this case, the personalized transfer function, which is a personalized model that defines a relationship between limb BCG signals and whole-body BCG signals, may be constructed in advance through various model construction schemes (e.g., machine learning, regression analysis, and the like) based on a user's limb BCG signal and whole-body BCG signal that are measured simultaneously and be stored in an internal or external database.

The signal segmenter 330 may generate a plurality of single-period signals by segmenting the transformed limb BCG signal by each period. In this case, the signal segmenter 330 may segment the transformed limb BCG signal by each period by analyzing a signal form of the transformed limb BCG signal itself, or segment the transformed limb BCG signal by each period based on a result of beat-gating of the limb BCG signal on the basis of another signal (e.g., ECG signal, photoplethysmogram (PPG) signal, and the like) measured simultaneously with the limb BCG signal.

The feature extractor 340 may extract characteristic points from the limb BCG signal segments. According to an exemplary embodiment, the feature extractor 340 may extract a maximum point and/or a minimum point of the limb BCG signal segment. For example, as shown in FIG. 4, the feature extractor 340 may extract G, H, I, J, K, and L as characteristic points from the limb BCG signal segment. The characteristic points may be inflection points in the graph of FIG. 4.

According to an exemplary embodiment, the feature extractor 340 may extract a characteristic point from each of the single-period signals, or determine a representative signal that represents limb BCG signals transformed based on a mutual similarity of a plurality of single-period signals and extract a characteristic point from the representative signal. For example, among the plurality of single-period signals, the feature extractor 340 may determine a single-period signal having the highest average similarity with other single-period signals as a representative signal, or determine an ensemble average of a predetermined number of single-period signals having a higher average similarity with other single-period signals as a representative signal. Alternatively, the feature extractor 340 may determine an ensemble average of two or more single-period signals having average similarities with other single-period signals greater than or equal to a predetermined threshold as a representative signal and then extract a maximum point and/or a minimum point of the determined representative signal as characteristic points. In this case, the feature extractor 340 may use various similarity calculation algorithms, such as Euclidean distance, Manhattan distance, cosine distance, Mahalanobis distance, Jaccard coefficient, extended Jaccard coefficient, Pearson's correlation coefficient, Spearman's correlation coefficient, and the like.

The feature extractor 340 may extract a blood pressure-related feature by combining time and/or amplitude of the extracted characteristic points. For example, referring to FIG. 4, the feature extractor 340 may extract time interval between points G and H, time interval between points G and I, time interval between points G and J, time interval between points G and K, time interval between points G and L, time interval between points H and I, time interval between points H and J, time interval between points H and K, time interval between points H and L, time interval between points I and J, time interval between points I and K, time interval between points I and L, time interval between points J and K, time interval between points J and L, time interval between points K and L, a proportion of these time intervals, an amplitude of point G, an amplitude of point H, an amplitude of point I, an amplitude of point J, an amplitude of point K, an amplitude of point L, and a proportion of these amplitudes as the blood pressure-related features.

The blood pressure estimator 350 may estimate a user's blood pressure on the basis of the extracted blood pressure-related features. In this case, the blood pressure estimator 350 may use a feature-blood pressure model that defines a relationship between the blood pressure-related feature and blood pressure. The feature-blood pressure model may be constructed in advance using various model construction schemes (e.g., machine learning, regression analysis, and the like) and be stored in an internal or external database.

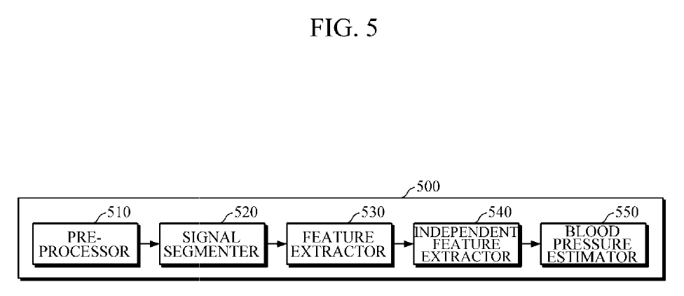

FIG. 5 is a block diagram illustrating a processor according to another exemplary embodiment. The processor 500 of FIG. 5 may be one exemplary embodiment of the processor 220 of FIG. 2.

Referring to FIG. 5, the processor 500 includes a preprocessor 510, a signal segmenter 520, a feature extractor 530, an independent feature extractor 540, and a blood pressure estimator 550.

The preprocessor 510 may remove noise from a limb BCG signal. In this case, the preprocessor 510 may remove noise from a limb BCG signal using various noise removal techniques, such as filtering, smoothing, and the like.

The signal segmenter 520 may generate a plurality of single-period signals by segmenting the limb BCG signal by each period. In this case, the signal segmenter 520 may segment the limb BCG signal by each period by analyzing a signal form of the limb BCG signal itself, or transform a limb BCG signal by each period based on a result of beat-gating of the limb BCG signal with respect to another signal (e.g., ECG signal, PPG signal, and the like) measured simultaneously with the limb BCG signal.

The feature extractor 530 may detect a maximum point (e.g., a local maximum amplitude) and/or a minimum point (e.g., a local minimum amplitude) from the limb BCG signal segment and extract the detected maximum point and/or minimum point as characteristic points. In addition, the feature extractor 530 may extract a blood pressure-related features based on time intervals between the extracted characteristic and/or amplitudes of the extracted characteristic points (e.g., by combining the times and/or amplitudes of the extracted characteristic points).

The independent feature extractor 540 may extract a feature independently associated with blood pressure (hereinafter, referred to as an “independent blood pressure-related feature”). In this case, the independent feature extractor 540 may extract the independent blood pressure-related feature using a dimensionality reduction method. The dimensionality reduction method may include, but not limited to, principal component analysis (PCA), independent component analysis (ICA), linear discriminant analysis (LDA), canonical correlation analysis (CCA), singular value decomposition (SVD), non-negative matrix factorization (NMF), locality preserving projection (LPP), margin preserving projection (MPP), Fisher linear discriminant (FLD), and the like.

The blood pressure estimator 550 may estimate a blood pressure of the user on the basis of the extracted independent blood pressure-related feature. In this case, the blood pressure estimator 550 may use an independent feature-blood pressure model that defines a relationship between the independent blood pressure-related feature and blood pressure. The independent feature-blood pressure model may be constructed in advance using various model construction schemes (e.g., machine learning, regression analysis, and the like) and be stored in an internal or external database.

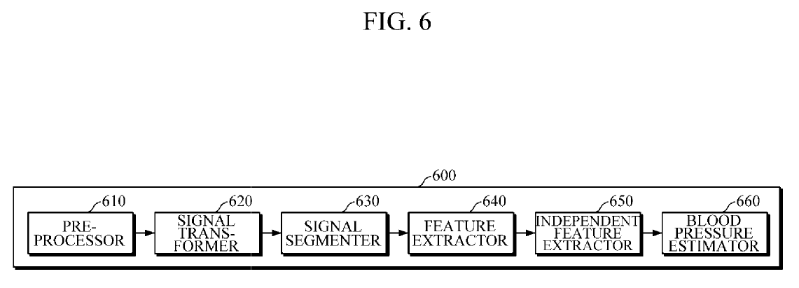

FIG. 6 is a block diagram illustrating a processor according to still another exemplary embodiment. The processor 600 of FIG. 6 may be one exemplary embodiment of the processor 220 of FIG. 2.

Referring to FIG. 6, the processor 600 includes a preprocessor 610, a signal transformer 620, a signal segmenter 630, a feature extractor 640, a feature extractor 640, an independent feature extractor 650, and a blood pressure estimator 660.

The preprocessor 610 may remove noise from a limb BCG signal. In this case, the preprocessor 610 may remove noise from the limb BCG signal using various noise removal techniques, such as filtering, smoothing, and the like.

The signal transformer 620 may transform the limb BCG signal into the form of a whole-body BCG signal. For example, the signal transformer 620 may transform the limb BCG signal into the form of whole-body BCG signal using a transfer function, such as an integrator or a differentiator, or a personalized transfer function.

The signal segmenter 630 may generate a plurality of single-period signals by segmenting the transformed limb BCG signal by each period.

The feature extractor 640 may extract a maximum point (e.g., a local maximum amplitude) and/or a minimum point (e.g., a local minimum amplitude) from the limb BCG signal segment as characteristic points. In addition, the feature extractor 640 may extract a blood pressure-related features based on time intervals between the extracted characteristic and/or amplitudes of the extracted characteristic points (e.g., by combining the times and/or amplitudes of the extracted characteristic points).

The independent feature extractor 650 may extract an independent blood pressure-related feature among the extracted blood pressure-related features. In this case, the independent feature extractor 650 may extract the blood pressure-related feature using a dimensionality reduction method.

The blood pressure estimator 660 may estimate user's blood pressure on the basis of the extracted independent blood pressure-related feature. In this case, the blood pressure estimator 660 may use an independent feature-blood pressure model that defines a relationship between the independent blood pressure-related feature and blood pressure.

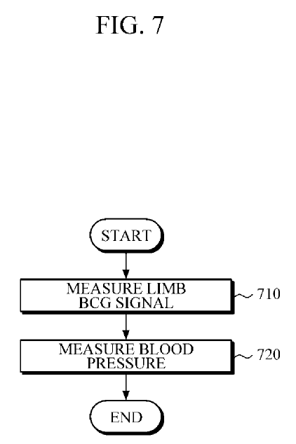

FIG. 7 is a flowchart illustrating a method of measuring blood pressure according to one exemplary embodiment. The method of measuring blood pressure of FIG. 7 may be performed by the apparatus 200 for measuring blood pressure of FIG. 2.

Referring to FIGS. 2 and 7, the apparatus 200 for measuring blood pressure may measure a limb BCG signal of a user in 710. To this end, the apparatus 200 may include various types of sensors, such as an acceleration sensor, a load cell sensor, a PVDF film sensor, and an EMFi sensor, and the like.

The apparatus 200 may extract a blood pressure-related feature by analyzing the measured limb BCG signal and estimate the user's blood pressure on the basis of all or part of the extracted blood pressure-related feature in 720.

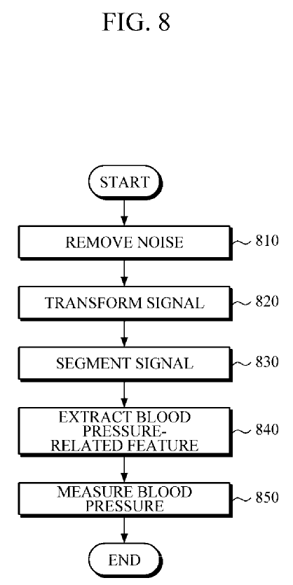

FIG. 8 is a flowchart illustrating a process 720 of estimating blood pressure according to one exemplary embodiment.

Referring to FIGS. 2 and 8, the apparatus 200 for measuring blood pressure may remove noise from a limb BCG signal in 810. In this case, the apparatus 200 may use various noise removal techniques, such as filtering, smoothing, and the like.

The apparatus 200 may transform the limb BCG signal into the form of a whole-body BCG signal in 820. For example, the apparatus 200 may transform the limb BCG signal into the form of whole-body BCG signal using a transfer function, such as an integrator or a differentiator, or a personalized transfer function.

The apparatus 200 may generate a plurality of single-period signal by segmenting the transformed limb BCG signal by each period in 830.

The apparatus 200 may extract characteristic points from the limb BCG signal segment and extract blood pressure-related features based on time intervals between the extracted characteristic and/or amplitudes of the extracted characteristic points (e.g., by combining times and/or amplitudes of the extracted characteristic points). According to one exemplary embodiment, the apparatus 200 may extract characteristic points from each of the single-period signals, or determine a representative signal that represents limb BCG signals transformed based on a mutual similarity of a plurality of single-period signals and extract characteristic points from the representative signal.

The apparatus 200 may estimate the user's blood pressure on the basis of the extracted blood pressure-related feature in 850. In this case, the apparatus 200 may use a feature-blood pressure model that defines a relationship between the blood pressure-related feature and blood pressure.

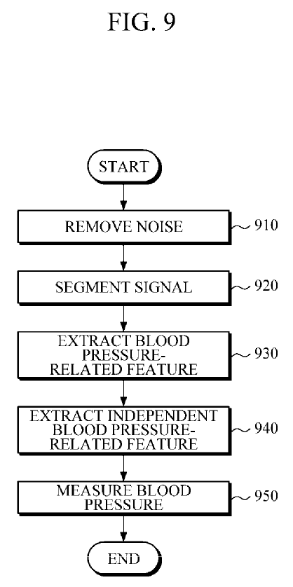

FIG. 9 is a flowchart illustrating a process 720 of estimating blood pressure according to another exemplary embodiment.

Referring to FIGS. 2 and 9, the apparatus 200 may remove noise from a limb BCG signal in 910. In this case, the apparatus 200 may use various noise removal techniques, such as filtering, smoothing, and the like.

The apparatus 200 may generate a plurality of single-period signals by segmenting the limb BCG signal by each period in 920.

The apparatus 200 may extract characteristic points from the limb BCG signal segment and extract blood pressure-related features by combining times and/or amplitudes of the extracted characteristic points in 930.

The apparatus 200 may extract an independent blood pressure-related feature among the extracted blood pressure-related features in 940. In this case, the apparatus 200 may use a dimensionality reduction method.

The apparatus 200 may estimate the user's blood pressure on the basis of the extracted independent blood pressure-related feature in 950. At this time, the apparatus 200 may use an independent feature-blood pressure model.

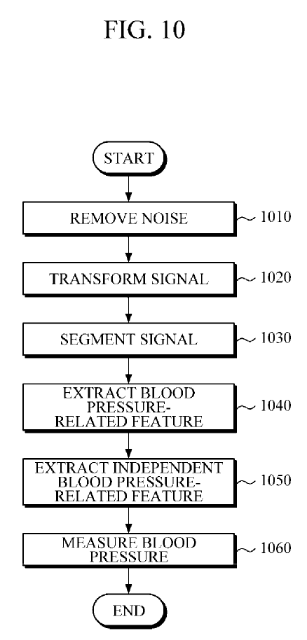

FIG. 10 is a flowchart illustrating a process 720 of estimating blood pressure according to still another exemplary embodiment.

Referring to FIGS. 2 and 10, the apparatus 200 may remove noise from a limb BCG signal in 1010. In this case, the apparatus 200 may use various noise removal techniques, such as filtering, smoothing, and the like.

The apparatus 200 may transform the limb BCG signal into the form of a whole-body BCG signal in 1020. For example, the apparatus 200 may transform the limb BCG signal into the form of whole-body BCG signal using a transfer function, such as an integrator or a differentiator, or a personalized transfer function.

The apparatus 200 may generate a plurality of single-period signals by segmenting the limb BCG signal by each period in 1030.

The apparatus 200 may extract characteristic points from the limb BCG signal segment and extract blood pressure-related features by combining times and/or amplitudes of the extracted characteristic points in 1040.

The apparatus 200 may extract an independent blood pressure-related feature among the extracted blood pressure-related features in 1050. In this case, the apparatus 200 may use a dimensionality reduction method.

The apparatus 200 may estimate the user's blood pressure on the basis of the extracted independent blood pressure-related feature in 1060. At this time, the apparatus 200 may use an independent feature-blood pressure model.

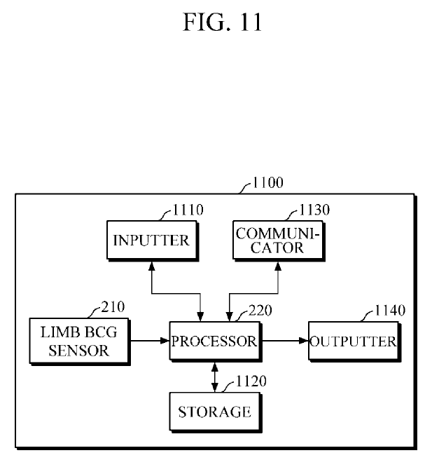

FIG. 11 is a block diagram illustrating an apparatus for measuring blood pressure according to another exemplary embodiment.

Referring to FIG. 11, an apparatus 1100 for measuring blood pressure includes a limb BCG sensor 210, a processor 220, an inputter 1110, a storage 1120, a communicator 1130, and an outputter 1140.

Here, the limb BCG sensor 210 and the processor 220 are the same as those described with reference to FIGS. 2 to 6, and hence detailed descriptions thereof will be omitted.

The inputter 1110 may receive various operation signals from a user. According to one exemplary embodiment, the inputter 1110 may include a keypad, a dome switch, a resistive or capacitive touch pad, a jog wheel, a jog switch, a hardware button, and the like. In particular, when a touch pad has a layered structure with a display, this structure may be referred to as a touch screen.

Programs or instructions for operations of the apparatus 1110 may be stored in the storage 1120 and data input to and output from the apparatus 1110 may also be stored in the storage 1120. In addition, data processed by the apparatus 1100 and data required by the apparatus 1100 to process data may be stored in the storage 1120.

The storage 1120 may include at least one type of storage media, such as a flash memory, a hard disk type memory, a multimedia card micro type memory, a card-type memory (e.g., Secure Digital (SD) or xD-Picture Card memory), random access memory (RAM), static random access memory (SRAM), read-only memory (ROM), electrically erasable programmable read-only memory (EEPROM), programmable read-only memory (PROM), magnetic memory, and optical disk. In addition, the apparatus 1100 may operate an external storage medium, such as web storage providing a storage function of the storage 1120.

The communicator 1130 may communicate with an external device. For example, the communicator 1130 may transmit data handled by the apparatus 1100 or processing result data of the apparatus 1100 to the external device or receive various pieces of data necessary or helpful for blood pressure estimation from the external device.

In this case, the external device may be medical equipment that uses the data handled by the apparatus 1100 or the processing result data of the apparatus 1100 or a printer or a display device to output a result. In addition, the external device may be a digital TV, a desktop computer, a mobile phone, a smartphone, a tablet computer, a notebook computer, a PDA, a PMP, a navigation system, an MP3 player, a digital camera, a wearable device, or the like, but is not limited thereto.

The communicator 1130 may communicate with the external device through various communication schemes, such as Bluetooth communication, Bluetooth low energy communication, near-field communication (NFC), wireless local area network (WLAN) communication, ZigBee communication, infrared data association (IrDA) communication, radio frequency identification communication, third generation (3G) communication, fourth generation (4G) communication, fifth generation (5G) communication, and the like. However, these are merely examples, and the communication scheme is not limited thereto.

The outputter 1140 may output the data handled by the apparatus 1100 or the processing result data of the apparatus 1100. According to one exemplary embodiment, the outputter 1140 may output the data handled by the apparatus 1100 or the processing result data of the apparatus 1100 in at least one of visual, audible, and tactile manners. To this end, the outputter 1140 may include a display, a speaker, a vibrator, and the like.

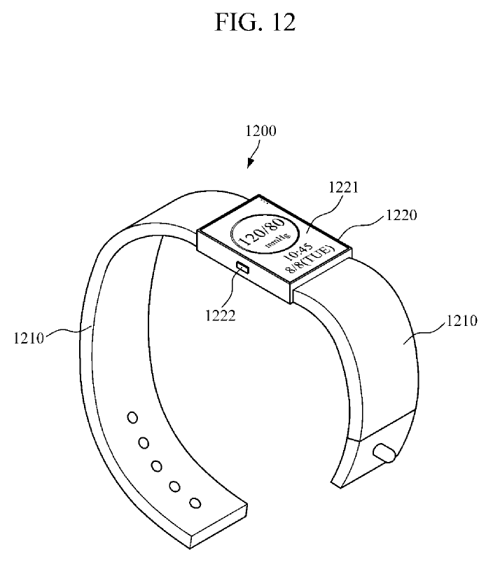

FIG. 12 is a diagram illustrating a wrist-wearable device.

Referring to FIG. 12, the wrist-wearable device 1200 includes a strap 1210 and a main body 1220.

The strap 1210 may be composed of separate strap members that are connected to each side of the main body 1220 and capable of being coupled to each other, or may be integrally formed in the form of a smart band. The strap 1210 may be formed of a flexible member to wrap around the user's wrist such that the main body 1220 can be worn on the user's wrist.

The above-described apparatus 200 or 1100 for measuring blood pressure may be equipped inside the main body 1220. In addition, a battery may be embedded in the main body 1220 to supply power to the wrist-wearable device 1200 and the apparatus 200 or 1100 for measuring blood pressure.

The wrist-wearable device 1200 may further include a display 1221 and an inputter 1222 which are mounted on the main body 1220. The display 1221 may display data processed by the wrist-wearable device 1200 and the apparatus 200 or 1100 for measuring blood pressure and processing result data. The inputter 1222 may receive various operating signals from the user.

The embodiments may be implemented as computer-readable code in a computer-readable record medium. Code and code segments constituting the computer program may be implemented by a skilled computer programmer in the art. The computer-readable record medium includes all types of record media in which computer-readable data are stored. Examples of the computer readable record medium include a ROM, a RAM, a compact disc ROM (CD-ROM), a magnetic tape, a floppy disk, and an optical data storage. Further, the record medium may be implemented in the form of a carrier wave such as Internet transmission. In addition, the computer-readable record medium may be distributed to computer systems over a network, in which computer-readable code may be stored and executed in a distributed manner.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An apparatus for measuring blood pressure, the apparatus comprising:
a limb ballistocardiogram (BCG) sensor configured to attach to a limb of a user and measure a limb BCG signal of the user; and
a processor configured to extract blood pressure-related features from the measured limb BCG signal and estimate a blood pressure of the user based on at least part of the extracted blood pressure-related features.

2. The apparatus of claim 1, wherein the limb BCG sensor comprises at least one of an acceleration sensor, a load cell sensor, a polyvinylidene fluoride (PVDF) film sensor, and an electro mechanical film (EMFi) sensor.

3. The apparatus of claim 1, wherein the processor comprises:
a signal transformer configured to transform the measured limb BCG signal into a form of a whole-body BCG signal;
a signal segmenter configured to segment the transformed limb BCG signal by each period to create a limb BCG signal segment;
a feature extractor configured to extract at least one of the blood pressure-related features from the limb BCG signal segment; and
a blood pressure estimator configured to estimate the blood pressure of the user based on the extracted at least one of the blood pressure-related features.

4. The apparatus of claim 3, wherein the signal transformer is further configured to transform the measured limb BCG signal into the form of the whole-body BCG signal using at least one of an integrator and a personalized model that defines a relationship between the limb BCG signal and the whole-body BCG signal.

5. The apparatus of claim 3, wherein the feature extractor is further configured to extract characteristic points from the limb BCG signal segment and extract the at least one of the blood pressure-related features based on at least one of time intervals between the extracted characteristic points and amplitudes of the extracted characteristic points.

6. The apparatus of claim 5, wherein the feature extractor is further configured to extract a maximum point and a minimum point of the limb BCG signal segment as the characteristic points.

7. The apparatus of claim 3, wherein the feature extractor is further configured to determine a representative signal that represents the transformed limb BCG signal using the limb BCG signal segment and extract the at least one of the blood pressure-related features from the determined representative signal.

8. The apparatus of claim 3, wherein the processor further comprises a preprocessor configured to remove noise from the measured limb BCG signal.

9. The apparatus of claim 1, wherein the processor comprises:
a signal segmenter configured to segment the measured limb BCG signal by each period to create a limb BCG signal segment;
a feature extractor configured to extract at least one of the blood pressure-related features from the limb BCG signal segment;
an independent feature extractor configured to extract at least one independent blood pressure-related feature from the extracted at least one of the blood pressure-related features; and
a blood pressure estimator configured to estimate the blood pressure of the user based on the extracted at least one independent blood pressure-related feature.

10. The apparatus of claim 9, wherein the independent feature extractor is further configured to extract the at least one independent blood pressure-related feature from the extracted at least one of the blood pressure-related features using a dimensionality reduction method.

11. The apparatus of claim 1, wherein the processor comprises:
a signal transformer configured to transform the measured limb BCG signal into a form of a whole-body BCG signal;
a signal segmenter configured to segment the transformed limb BCG signal by each period to create a limb BCG signal segment;
a feature extractor configured to extract at least one of the blood pressure-related features from the limb BCG signal segment;
an independent feature extractor configured to extract at least one independent blood pressure-related feature from the extracted at least one of the blood pressure-related features; and
a blood pressure estimator configured to estimate the blood pressure of the user based on the extracted at least one independent blood pressure-related feature.

12. A method of measuring blood pressure, the method comprising:
measuring a limb ballistocardiogram (BCG) signal of a user;
extracting blood pressure-related features from the measured limb BCG signal; and
estimating a blood pressure of the user based on at least part of the extracted blood pressure-related features.

13. The method of claim 12, wherein the extracting the blood pressure-related features comprises:
transforming the measured limb BCG signal into a form of a whole-body BCG signal;
segmenting the transformed limb BCG signal by each period to create a limb BCG signal segment;
extracting at least one of the blood pressure-related features from the limb BCG signal segment; and
estimating the blood pressure of the user based on the extracted at least one of the blood pressure-related features.

14. The method of claim 13, wherein the transforming the measured limb BCG signal comprises transforming the measured limb BCG signal into the form of the whole-body BCG signal using at least one of an integrator and a personalized model that defines a relationship between the limb BCG signal and the whole-body BCG signal.

15. The method of claim 13, wherein the extracting the at least one of the blood pressure-related features comprises extracting characteristic points from the limb BCG signal segment and extracting the at least one of the blood pressure-related features based on at least one of time intervals between the extracted characteristic points and amplitudes of the extracted characteristic points.

16. The method of claim 15, wherein the extracting the characteristic points comprises extracting a maximum point and a minimum point of the limb BCG signal segment as the characteristic points.

17. The method of claim 13, wherein the extracting the at least one of the blood pressure-related features comprises determining a representative signal that represents the transformed limb BCG signal using the limb BCG signal segment and extracting the at least one of the blood pressure-related features from the determined representative signal.

18. The method of claim 12, wherein the extracting the blood pressure-related features comprises:
segmenting the measured limb BCG signal by each period to generate a limb BCG signal segment;
extracting at least one of the blood pressure-related features from the limb BCG signal segment; and
extracting at least one independent blood pressure-related feature from the extracted at least one of the blood pressure-related features; and
estimating the blood pressure of the user based on the extracted at least one independent blood pressure-related feature.

19. The method of claim 18, wherein the at least one independent blood pressure-related feature is extracted using a dimensionality reduction method.

20. The method of claim 12, wherein the extracting the blood pressure-related features comprises:
transforming the measured limb BCG signal into a form of a whole-body BCG signal;
segmenting the transformed limb BCG signal by each period to create a limb BCG signal segment;
extracting at least one of the blood pressure-related features from the limb BCG signal segment; and
extracting at least one independent blood pressure-related feature from the extracted at least one of the blood pressure-related features.
